(19) United States
(12) Patent Application Publication
(10) Pub. No.: US 2014/0322807 A1
(43) Pub. Date: Oct. 30, 2014

(54) METHOD FOR EXTRACTING NUTRIENTS FROM ORGANIC MATERIALS

(71) Applicant: Innovative Bios L.L.C., Wilmington, DE (US)

(72) Inventors: John Paul Gustin, Baltimore, MD (US); Jason Allen Repac, Wilmington, DE (US)

(73) Assignee: INNOVATIVE BIOS L.L.C., Wilmington, DE (US)

(21) Appl. No.: 14/360,176

(22) PCT Filed: Dec. 6, 2012
(86) PCT No.: PCT/US2012/068234

§ 371 Date: May 22, 2014

Related U.S. Application Data
(60) Provisional application No. 61/567,538, filed on Dec. 6, 2011.

(51) Int. Cl. C12N 5/00; C12N 1/12; C12N 1/16; C12N 1/20

(52) U.S. Cl. CPC C12N 5/0018; C12N 1/20; C12N 1/12; C12N 1/16
USPC 435/348; 435/253.6; 435/257.1; 435/255.7; 435/431; 435/404

(57) ABSTRACT

Processes for extracting and recovering nutrients from organic wastes to create a cell culture broth for microorganisms involve the main steps of mixing, solid/liquid separation, optimization, and sterilization. In an embodiment, the method for converting organic waste material into a cell culture broth or growth media includes: (a) mixing an organic waste material with one or more solvent to create a mixture of liquids and solids under substantially turbulent conditions; (b) separating the mixture of liquids and solids into a liquid stream and solid stream; and (c) sterilizing the liquid stream, whereby the cell culture broth or growth media comprises the sterilized liquid stream.

### CROSS-REFERENCE TO RELATED APPLICATIONS

This application claims the benefit of U.S. Provisional Application No. 61/567,538, filed Dec. 6, 2011, the content of which is incorporated by reference in its entirety.

### FIELD OF THE INVENTION

The present invention generally relates to processes for extracting and recovering nutrients from organic wastes and for converting these organic wastes into cell culture broth or growth media.

### BACKGROUND OF THE INVENTION

Currently, the majority of cell culture broths are produced in the following manner. Organic and inorganic sources of nutrients are obtained from various sources. These nutrients undergo analytical testing before being separated into organic and inorganic components. The inorganic components undergo weighing and either proceed to (a) mixing and grinding or (b) granulation and sieving to achieve the desired nutrient profile. The organic compounds are then separated into definite products, such as sugars, indicators, and inhibitors, or indefinite products, such as peptones, extracts, and gelifiers. The definite products are then separated by proportion into minority or massive components. The minority components are weighed and then are mixed and ground. The massive organic components undergo weighing, then granulation and sieving. The indefinite products are then weighed. All the inorganic and organic components, such as definite minority and massive components and indefinite organic components, are then mixed and sieved before undergoing analytical controls in which the physical, chemical, and microbiological properties of the resulting mixture is analyzed for the desired properties, before being packaged for sale. The mixture may optionally be dissolved in water and sterilized in some fashion before undergoing packaging.

The method described above may be inadequate because the demand for the nutrients is increasing due to increased agricultural production and increased use of microorganisms as production platforms while the supply of some of these nutrients is decreasing. This is predicted to result in increased scarcity and prices for these nutrients. This may be especially true for phosphorous, of which production is nearing its peak and prices have been rising substantially over recent years.

At the same time, organic waste such as those produced by humans, livestock, and plants contain a large number of nutrients. Currently, processing of organic wastes may preferably be carried out by using as little energy as possible. These processes may be used to create relatively low value products. For instance, currently, most organic waste such as e.g. animal waste is used as fertilizer. However, there is always an excess of waste when compared to local on-farm needs. For example, there is an estimated 100,000 tons per year of excess poultry litter on Maryland's Eastern Shore. This may present a problem since over-application of manure can lead to eutrophication and pollution of surface waters. Treatment of excess animal waste requires costly transportation of tons of animal waste for long distances.

As such, organic waste may provide a renewable, inexpensive, and sustainable source of these nutrients in the production of a cell culture broth. However, organic waste may need to be processed further to be able to utilize the nutrients. Thus far, there has been no easy to use method to process organic waste into a culture broth or media.

### SUMMARY OF THE INVENTION

Embodiments relate to processes for converting organic waste into a cell culture broth. These processes can include the addition and/or removal of substances to optimize the propagation, culture, or fermentation of bacteria, algae, yeast, synthetically derived microorganisms, insect cells, plant cells, or mammalian cells as well as the sterilization of the broth. The broth may be produced in either liquid or anhydrous form. The processes may have an additional solid waste stream that may be utilized for other purposes such as e.g. fertilization of plants, anaerobic digestion, direct combustion, gasification, or pyrolysis.

One embodiment is a method for converting organic waste material into a cell culture broth or growth media including: mixing an organic waste material with one or more solvent to create a mixture of liquids and solids; separating the mixture of liquids and solids into a liquid stream and solid stream; and sterilizing the liquid stream, whereby the cell culture broth or growth media comprises the sterilized liquid stream.

The cell culture broth or growth media may be a 1× solution, concentrated solution, or anhydrous form. The concentrated solution may contain all or some of the nutrients and chemicals for optimal growth conditions of bacteria, algae, yeast, synthetically derived microorganisms, insect cells, plant cells, or mammalian cells. The cell culture broth and/or growth media may have a variety of uses and may be suitable for the propagation, culture, fermentation, or maintenance of bacteria, algae, yeast, synthetically derived microorganisms, insect cells, plant cells, or mammalian cells. Furthermore, the cell culture broth and/or growth media may be provided as part of a composition and/or kit. Optionally, the cell culture broth or growth media may be supplemented to achieve optimal growth conditions of bacteria, algae, yeast, synthetically derived microorganisms, insect cells, plant cells, or mammalian cells.

The organic waste material may be livestock manure, bedding, litter, human organic waste, plant waste, and mixtures thereof. Furthermore, the organic waste material may be processed or unprocessed. In an embodiment, the organic waste material is processed by e.g. milling, composting, pyrolysis, autoclaving, gasification, anaerobic digestion, combustion, autoclaving, and combinations thereof. In another embodiment, the organic waste material is plant waste, which optionally may be processed. The plant waste is preferably free of animal waste. In yet another embodiment, the plant waste is free of livestock manure, bedding, litter, and human organic waste.

The step of mixing organic waste material with one or more solvent to create a mixture of liquids and solids may be conducted via a continuous or batch process, optionally with heating. The step of mixing may preferably also be carried out under substantially turbulent, turbulent, or transitional conditions. The step of mixing may also be carried out under conditions having a Reynolds number (Re) of greater than 2000. A variety of solvents may be used. For example, the one or more solvent may be water, alcohol or an organic solvent. The step of mixing may include agitation, such as e.g. agitation for about 30 to 60 minutes at about 150 to 200 rpm. The step of mixing may also include a residence time from about 15 minutes to about 3 hours and heating such as e.g. heating up to about 100 degrees C.

The step of separating the mixture of liquids and solids into a liquid stream and solid stream may include filtration, gravitational settling, decanting, centrifugation, or combinations thereof. According to an embodiment, the liquid stream contains nutrients, and one or more solvent.

The step of sterilizing the liquid stream can include filtration, irradiation, chemical treatment, heating, pressurization, or combinations thereof. In an embodiment, the step of sterilizing the liquid stream includes heating the liquid stream to a temperature of about 121 degrees C. at a pressure of about 15 psig and maintaining these conditions for at least about 15 minutes.

The method may further include a variety of other method steps. For example, the method further may include analyzing the organic waste material for chemical composition prior to mixing, analyzing the liquid stream and the solid stream for chemical composition after separating the mixture of liquids and solids into a liquid stream and solid stream and optimizing the composition of the liquid stream depending on the desired use of the cell culture broth or growth media. The step of optimizing may include one or more of (a) adding nutrients or chemicals to the liquid stream, (b) removing and/or deactivating excess nutrients or chemicals from the liquid stream, (c) removing and/or deactivating antibiotics and other growth harming substances from the liquid stream and combinations thereof. According to an embodiment, the step of optimizing increases the growth rate of bacteria, algae, yeast, synthetically derived microorganisms, insect cells, plant cells, or mammalian cells in the cell culture broth or growth media. The method may also include the step of selecting one or more solvent based on the chemical composition of the organic waste material prior to mixing the organic waste material with the one or more solvent. Depending on the desired use, all or some of these method steps may be combined.

The solid stream may also be further processed. In an embodiment, the method also includes anaerobic digestion of the solid stream, gasification of the solid stream, combustion of the solid stream, pyrolysis of the solid stream or combinations thereof. The solid stream may also be reused in the process by mixing the solid stream with the organic materials. The solid stream may also be suitable for use as a plant fertilizer. In an embodiment, the methods further include anaerobic digestion of the solid stream and reusing the solid stream in the process by mixing the solid stream with organic materials.

In another embodiment, the methods can further include one or more of the following steps: adding nutrients or chemicals to the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells without the nutrients or chemicals; removing and/or deactivating excess nutrients or chemicals from the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells with the nutrients or chemicals; removing and/or deactivating antibiotics and other growth harming substances from the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells with the antibiotics and other growth harming substances; or combinations thereof.

The method may further include one or more of the following steps: analyzing organic waste material for chemical composition; analyzing liquid stream for chemical composition; analyzing solid stream for chemical composition or the combination thereof. A variety of analytical techniques may be used to analyze the organic waste material, liquid stream, and/or solid stream such as e.g. mass spectrometry, Fourier Transform Infrared Spectrometry and High Pressure Liquid Chromatography.

Other features and advantages will be apparent from the detailed description and examples that follow.

### BRIEF DESCRIPTION OF THE DRAWINGS

The foregoing summary, as well as the following detailed description of the invention, will be better understood when read in conjunction with the appended figure. For the purpose of illustrating the invention, the figure demonstrates embodiments of the present invention. It should be understood, however, that the invention is not limited to the precise arrangements, examples, and instrumentalities shown.

FIG. 1 shows a flowchart of the method steps performed in an embodiment of the present invention.

### DETAILED DESCRIPTION OF INVENTION

### I. Definitions

Various terms used throughout the specification and claims are defined as set forth below. In the present disclosure, the singular forms “a,” “an,” and “the” include the plural reference and reference to a particular numerical value includes at least that particular value, unless the context clearly indicates otherwise. When values are expressed as approximations, by use of the antecedent “about,” it will be understood that the particular value forms another embodiment. Where present, all ranges are inclusive and combinable.

As used herein, the term “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “livestock” refers to any animal kept by humans for a useful, rather than recreational purpose. Exemplary livestock animals include but are not limited to cattle, sheep, pigs, goats, horses, donkeys, mules, buffalo, oxen, or camels. As used herein, the term livestock also encompasses poultry such as e.g. chicken, duck, turkey, goose, pheasants, pigeons, and the like.

“Cell culture broth” or “growth media” are any liquid/anhydrous powder used for the propagation, culture, fermentation, or maintenance of bacteria, algae, yeast, synthetically derived microorganisms, insect cells, plant cells, or mammalian cells.

“1× solution” is a liquid cell culture broth or growth media that does not need to be diluted and can be used as is.

“Bacteria” are any prokaryote microorganisms, which can be naturally occurring, synthetically derived, or genetically manipulated.

“Algae” are any prokaryotic and eukaryotic algae, which can be naturally occurring, synthetically derived, or genetically manipulated. This includes both unicellular and multicellular algae.

“Yeast” are any eukaryotic yeast, which can be naturally occurring, synthetically derived, or genetically manipulated.

“Synthetically” derived microorganisms are any organism created using synthetic biology.

“Growth harming substances” are any organic or inorganic compounds that may decrease the growth rate of or increase cell death of bacteria, algae, yeast, synthetically derived microorganisms, insect cells, plant cells or mammalian cells by about 0.5% or more, preferably about 0.25% or more, more preferably about 0.10% or more, more preferably about 0.05% or more.

“Optimization of growth rate” is the addition, removal, or modification of any substances contained in the cell culture broth that may increase the growth rate or decrease the cell death of bacteria, algae, yeast, synthetically derived microorganisms, insect cells, plant cells or mammalian cell by about 0.1% or more, preferably about 0.5% or more, more preferably about 1% or more, more about preferably 10% or more.

A “turbulent condition” refers to a flow regime characterized by chaotic and stochastic property changes. This includes low momentum diffusion, high momentum convection, and rapid variation of pressure and velocity in space and time. This turbulent condition is usually characterized as having a large Reynolds Number.

A “substantially turbulent condition” is a condition that is approximately turbulent. As used herein the term substantially turbulent shall encompass the laminar-turbulent transition condition as well as the turbulent condition.

A “transitional condition” refers to a flow regime in the laminar-turbulent transition.

### II. Description

The principles, preferred embodiments, and modes of operation of the present invention are described hereunder. The invention disclosed herein should not, however, be construed as limited to the particular forms disclosed, as these are to be regarded as illustrative rather than restrictive. Variations and changes may be made by those skilled in the art without departing from the spirit of the present invention. Accordingly, the examples, descriptions, and mode of carrying out the invention described herein should be considered exemplary in nature and not as limiting to the scope and spirit of the invention as set forth in the claims.

Embodiments of the invention provide for methods of producing an economical and renewable cell culture broth or media that can be used in the biotechnology and biofuels industry. In particular, these embodiments provide for processes for extracting and recovering nutrients from organic wastes to create a cell culture broth for microorganisms. The processes can involve the main steps of (1) mixing, (2) solid/liquid separation, (3) optimization, and (4) sterilization. Additional steps may be added as necessary. Unlike current processes of utilizing organic wastes, which typically may require as little energy as possible because the ultimate purpose of the process may be to provide a low value product (such as e.g. fertilizer) and to dispose of the waste, the invention provides for processes that may require more energy but that are capable of creating a high value product. Therefore, higher costing processes, such as autoclaving, can be profitably used since cell culture broths have a higher per unit value than other low value products made from organic wastes.

Embodiments of the invention thus provide for ways to create culture broth while utilizing wastes thereby reducing the amount of organic waste and providing a cheap and readily obtainable nutrient source for culture broths.

Accordingly, one aspect of the invention is a method converting of organic waste materials into a cell culture broth or growth media including the steps of mixing organic waste material with solvent to create a mixture of liquids and solids; separating the mixture of liquids and solids into a liquid stream and solid stream; and sterilizing the liquid stream. Additional other steps may be added if necessary and are discussed more in detail below. For example, in an embodiment, the method may further include the step of providing the organic waste materials.

The resultant cell culture broth or growth media includes the components of the sterilized liquid stream. In addition, resultant cell culture broth or growth media may be a 1× solution, concentrated solution, or anhydrous form. The culture broth or growth media has a variety of contemplated uses including but not limited to propagation, culture, fermentation, or maintenance of bacteria, algae, yeast, synthetically derived microorganisms, insect cells, plant cells, or mammalian cells. Preferably, the culture broth or growth media contains all or some of the nutrients and chemicals for optimal growth conditions of bacteria, algae, yeast, synthetically derived microorganisms, insect cells, plant cells, or mammalian cells. As described more in detail below, where necessary the culture broth or growth media may be supplemented.

The organic waste material may be from a variety of sources. In an embodiment, the organic waste material is livestock manure, bedding, litter, and human organic waste, plant waste, and mixtures thereof. The organic waste may be provided in unprocessed or processed form. Such processing includes but is not limited to as milling, composting, pyrolysis, autoclaving, gasification, anaerobic digestion, combustion, etc. prior to use of the material in the process. In an embodiment, the organic waste may be processed or unprocessed plant waste. The use of plant waste may be beneficial in situations where animal-based cell culture broths are not suitable for example because of safety and/or contamination concerns. Thus, in an embodiment, the organic waste material is solely plant waste, which is substantially free or more preferably free of animal (e.g. livestock or human) organic waste material. In another embodiment, the plant waste is free of livestock manure, bedding, litter, and human organic waste.

The step of mixing organic waste material with solvent to create a mixture of liquids and solids may be conducted via a continuous or batch process via heating. In an embodiment, the step of mixing may be conducted with heating. The step of mixing may preferably be carried out under substantially turbulent conditions. In an embodiment, the step of mixing is carried out under turbulent conditions. In another embodiment, the step of mixing is carried out under transitional conditions. The step of mixing may rely on convection. Furthermore, the step of mixing may be achieved without relying on diffusion alone. In an embodiment, the step of mixing may be carried out in a continuous plug flow reactor (PFR) with static mixer. In another embodiment, step of mixing may be carried out in a continuous stirred tank reactor (CSTR). Those of skill in the art would recognize that many of the parameters for the mixing may vary since many parameters are scale dependent. For example, stirrer rpm varies with reactor size.

Applicants have found that the processes of the invention, in particular the mixing may be optimally achieved when the mixing conditions have a Reynolds number of greater than 2000. In fluid mechanics, the Reynolds number is a dimensionless number that gives a measure of the ratio of inertial forces to viscous forces and consequently quantifies the relative importance of these two types of forces for given flow conditions. They are used to characterize different flow regimes, such as laminar or turbulent flow: laminar flow occurs at low Reynolds numbers, where viscous forces are dominant, and is characterized by smooth, constant fluid motion; turbulent flow occurs at high Reynolds numbers and is dominated by inertial forces, which tend to produce chaotic eddies, vortices and other flow instabilities. The Reynolds number is defined as follows:

$$Re = \frac{ρ vL}{μ} = \frac{vL}{ν}$$

where:

V is the mean velocity of the object relative to the fluid (m/s);

L is a characteristic linear dimension (m);

μ is the dynamic viscosity of the fluid (Pa·s or N·s/m² or kg/(m·s));

ν is the kinematic viscosity (ν=μ/ρ) (m²/s); and

ρ is the density of the fluid (kg/m³).

The step of separating the mixture of liquids and solids into a liquid stream and solid stream may include filtration, gravitational settling, decanting, centrifugation, or combinations thereof. The liquid stream may contain a variety of materials including but not limited to nutrients and one or more solvent. The liquid stream may be further processed.

Thus, the methods of the invention may include a variety of other method steps after obtaining the liquid stream. In an embodiment, the method may further comprise adding nutrients or chemicals to the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells without the nutrients or chemicals. In another embodiment, the method may further comprise removing and/or deactivating excess nutrients or chemicals from liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells with the nutrients or chemicals. In yet another embodiment, removing and/or deactivating antibiotics and other growth harming substances from the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells with the antibiotics and other growth harming substances. In an alternate embodiment, the method may further comprise one or more of these additional steps.

The solid stream may have a variety of uses such as e.g. fertilizing plants, anaerobic digestion, reusing in the process, gasification, combustion, pyrolysis, or combinations thereof. In an embodiment, solid stream may be anaerobically digested and then may be added to additional organic waste as the process is run again. Alternatively, the solid stream byproducts may be used instead of organic waste as the process is run again.

The method may further include one or more analysis step. Accordingly, in an embodiment, the method further includes analyzing the organic waste material for chemical composition. In another embodiment, the method further comprises analyzing the liquid stream for chemical composition. In yet another embodiment, the method further includes analyzing the solid stream for chemical composition. In another embodiment, the method further comprises a combination of each of these steps.

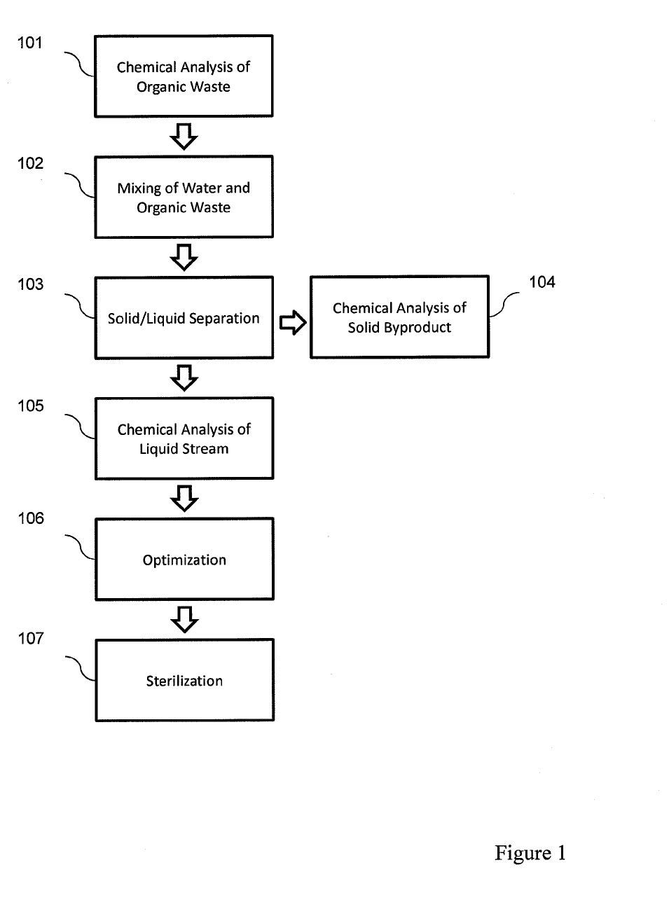

An exemplary embodiment of the process is depicted in FIG. 1. FIG. 1 shows a flowchart of the steps performed in an embodiment of the present invention. With reference to FIG. 1, the process generally encompasses chemical analysis of organic waste 101 followed by mixing of water and organic waste 102. After the mixing, the solids and liquids are separated in solid/liquid separation 103. Optionally, chemical analysis of solid byproduct(s) 104 may be conducted. After liquid separation, chemical analysis of liquid stream 105 is performed. The process is then optimized in optimization step 106. Ultimately, the recovered nutrients are sterilized in sterilization step 107.

As shown in FIG. 1, the method begins at the chemical analysis of organic waste step 101, where the organic wastes, such as e.g. livestock manure, are analyzed for their chemical composition. The analysis may be carried out utilizing for example mass spectrometry, Fourier Transform Infrared Spectroscopy (FTIR), High Pressure Liquid Chromatography (HPLC) and/or any other suitable method in accordance with the knowledge of one having ordinary skill in the art. Based on this measured composition, an operator may make an estimation of the final chemical compositions of the solid and liquid phases that will be produced following the method disclosed herein. This may determine what chemicals, nutrients, or substances, if any, may be needed to be added, removed, or deactivated during the optimization step 106. For example, in an embodiment, the amount of phosphate present may be at a 1 to 1 ratio compared to the solubility limit in water.

In an embodiment, following this analysis, the determination of the proper solvent (e.g. water) to organic waste ratio may be achieved. Since nitrate, phosphates, glucose, and peptides are present at a certain ratio, the organic waste may be mixed with the solvent (e.g. water) based on the limiting nutrient. For example, the amount of phosphate present in the organic waste may be at an about 10 to 1, alternatively about 20 to 1, alternatively about 30 to 1, alternatively about 40 to 1, alternatively about 50 to 1 ratio compared to the desired nutrient profile for a dilute broth.

Also compositional analysis allows the operator to determine which microorganism the end product of the liquid stream, i.e., a cell culture broth or growth media, may best be used to grow and how best to utilize the solid byproduct stream, for example the fertilization of plants, anaerobic digestion, or pyrolysis. This determination may be made by considering relevant chemical and physical properties of the organic waste, for example pH, carbon composition, nitrogen composition, and phosphorous composition. However, it is within the scope of the invention that any relevant property may be used in accordance with the knowledge of one having ordinary skill in the art.

The method proceeds to the mixing of water and organic waste step 102, where the organic wastes are then mixed with water or some other solvent, such as e.g. an alcohol, another organic solvent, or combinations thereof, to extract the nutrients, such as nitrates, phosphates, peptides, and sugars. This may be done in a batch or continuous fashion, optionally with heating. Agitation, such as stirring or shaking, may be added as to increase the rate of extraction. Agitation should be achieved by mixing in the turbulent flow regime. Agitation may, thus, be achieved by mixing under (1) substantially turbulent, (2) turbulent or (3) transitional conditions.

Following a residence time (for example, 15 minutes to 3 hours), the method proceeds to the solid/liquid separation step 103, where the organic waste/water mixture are separated into liquids and solids. Exemplary suitable residence times range from between about 15 minutes to about 3 hours, alternatively from about 20 minutes to about 2 hours, alternatively from about 1 hour to about 3 hours. The residence time may vary depending on the chemical composition of the organic waste.

Those of skill in the art would recognize that extraction conditions (i.e. mixing and separating conditions) may depend on waste nutrient profiles and desired product profiles. For example, the pH of the extraction conditions may be altered to increase extraction efficiency of certain compounds and retard others. In an embodiment, heating increases solubility limits of the solution but can lead to decomposition of some desired nutrients.

In an embodiment, the step of mixing comprises agitation for about 30 to 60 minutes at about 150 to 200 rpm under turbulent conditions. Such agitation may be carried out at room temperature. Alternatively, the mixing may be carried out at an elevated temperature. Optionally, it may be heated up to about 100 degrees C. in order to aid in solubilizing the nutrients in water. Those of skill in the art will recognize that agitator RPMs are reactor design and size dependent. Thus, at larger reactor sizes lower rpms may be used in order to achieve turbulent mixing. At small reactor sizes, higher RPMs may be needed.

The solid/liquid separation step 103 may be carried out by a variety of techniques well known in the art. For example, this separation could be carried out by a combination of filtration, gravitational settling, and/or decanting. Alternatively, one or more of these techniques could be used alone.

In an embodiment, the solids are allowed to gravitationally settle for 30 to 60 minutes after which the product liquid medium may be decanted leaving the solids in the reactor.

The solid byproduct stream can be chemically characterized at the chemical analysis of solid byproduct step 104. This solid byproduct stream contains insoluble materials as well as soluble materials that were not completely dissolved during the process. Based on this composition, a determination of how best to utilize this solid byproduct stream will be made. For example, the solid byproduct stream may be used for fertilizing plants, anaerobic digestion, gasification, combustion, pyrolysis, reused in the process, or combinations thereof.

The solid byproduct stream may go through anaerobic digestion after analysis and then be added to the organic waste either before step 101 or step 102. Alternatively, after anaerobic digestion, the solid byproduct may be used instead of the organic waste. Thus, the method may also include chemical analysis of the solid byproduct in step 101 and mixing step 102.

Following separation, the method proceeds to the chemical analysis of liquid stream step 105, where the liquid stream is analyzed for its chemical composition. Common liquid analysis techniques may be used. For example, the analysis may be done by mass spectrometry, Fourier Transform Infrared Spectroscopy (FTIR), High Pressure Liquid Chromatography (HPLC), and/or any other relevant method in accordance with the knowledge of one having ordinary skill in the art.

Based on this composition, the operator can make an exact determination of what chemicals, nutrients, or substances, if any, may be needed to be added, removed, or deactivated at the optimization step 106. The optimization step 106 may be highly dependent on the organic waste composition and the desired culture composition for each specific organism and will vary accordingly.

In an aspect of the present invention, the process then may optionally add and/or remove substances to optimize the composition in order to achieve the desired characteristics to promote cell growth at the desired level. These additions may include one or more carbon source, one or more metal scavengers to remove undesired metals, one or more pH buffers, and any other chemical/compound that one skilled in the art would know are suitable to optimize cell growth. For example, a carbon source may be suitable to optimize a bacteria cell culture broth, but would not necessarily be needed for an autotrophic algal cell culture broth. Another example may include the addition of a pH buffer to achieve and maintain the desired pH for the optimal growth of a specific organism.

In an embodiment, the appropriate additives are added to optimize the broth to comply with growth media specifications such as raising the pH with pH buffers, removing heavy metals with chelators, or adding glucose source, to provide energy for example, to the microorganism. The liquid medium may then be processed through a pre-filter and fine filter to remove any undesired solids.

In an aspect of the present invention after the optimization step 106, the method proceeds to the sterilization step 107. The sterilization step 107 allows for the destruction of biological entities, such as microorganisms and viruses, and may allow for in some cases the removal/deactivation of growth harming substances.

In an embodiment, the broth is sterilized via autoclave. For example, the broth may be heated to about 121 degrees C. at 15 psig and held for at least 15 minutes to ensure sterilization. In another embodiment, the broth may sterilized by ultraviolet (UV) irradiation. For example, the broth may be exposed to UV light with a wavelength of 254 nm for a prolonged residence time of over an hour to kill about 99.9999% of microorganisms.

In another aspect of the present invention, the sterilization step 107 may occur prior to the optimization step 106. The sterilization step 107 may include, but is not limited to, filtration, irradiation, chemical treatment, pressurization, heating, or combinations thereof. In another aspect of the present invention, the optimization step 106 may be omitted and the method may proceed directly from the chemical analysis of liquid stream step 105 to the sterilization step 107. The product of the above steps is a cell culture broth or growth media for the propagation, culture, or fermentation of bacteria, algae, yeast, synthetically derived microorganisms, insect cells, plant cells, or mammalian cells. It can then be packaged and/or used as is, diluted, or dehydrated.

After sterilization, the product (i.e. the cell culture broth or growth media) is ready for packaging and distribution following a final analytical analysis to ensure quality compliance. The solid byproduct left in the reactor may be dried and may be used in a pyrolysis process to provide the heat for the autoclave. Alternatively, the solid byproduct may be used for fertilizing plants, anaerobic digestion, gasification, combustion, pyrolysis, reused in the process, or combinations thereof.

In an embodiment, the methods using the disclosed process parameters may be used to process between about 1,000 tons to about 100,000 tons of waste material/year.

The invention also encompasses kits comprising the culture broth or growth media. In an embodiment, the kits can comprise the culture broth or growth media and instructions for growing (depending on the culture broth or growth media) bacteria, algae, yeast, synthetically derived microorganisms, insect cells, plant cells, and/or mammalian cells.

Without further description, it is believed that one of ordinary skill in the art can, using the preceding description and the following illustrative examples, make and utilize the present invention and practice the claimed methods. The following examples, therefore, specifically point out embodiments of the present invention, and are not to be construed as limiting in any way the remainder of the disclosure.

### PROPHETIC EXAMPLE 1

### Cell Culture Broth/Media from Livestock Manure

First, a sample of the source livestock manure may be analyzed to determine in what concentration it should be combined with water. Following this analysis, the determination of the proper water to manure ratio can be achieved. Because nitrate, phosphates, glucose, and peptides are present at a certain ratio, the manure will be mixed with water based on the limiting nutrient. For example, the amount of phosphate present in the manure may be at an about 20 to 1 ratio compared to the solubility limit in water. This would mean the operator would combine about 5000 liters of water and about ¼ ton of cow manure in a 10000-liter batch reactor based on the limiting nutrient composition. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the agitation may be turned off and the solids are allowed to gravitationally settle for about 30 to 60 minutes. The product liquid medium may be decanted leaving the solids in the reactor. The liquid medium may be analyzed for nutrient deficiencies or unwanted nutrients. The appropriate additives are added to optimize the broth to comply with growth media specifications such as raising the pH with pH buffers, removing heavy metals with chelators, or adding glucose source, to provide energy for the microorganism. The liquid medium may then be processed through a pre-filter and fine filter to remove any undesired solids. Once the broth is optimized, it can be sterilized via autoclave. The broth may be heated to about 121 degrees C. at 15 psig and held for at least 15 minutes to ensure sterilization. After the above steps, the product, the cell culture broth or growth media, is ready for packaging and distribution following a final analytical analysis to ensure quality compliance. The solid byproduct left in the reactor may be dried and may be used in a pyrolysis process to provide the heat for the autoclave.

### PROPHETIC EXAMPLE 2

### Cell Culture Broth/Media from Chicken Manure

First, a sample of the source chicken manure may be analyzed to determine in what concentration it should be combined with water. Following this analysis, the determination of the proper water to manure ratio can be achieved. Based on the fact that nitrate, phosphates, glucose, and peptides are present at a certain ratio, the manure will be mixed with water based on the limiting nutrient. For example, the amount of phosphate present in the manure may be at an about 20 to 1 ratio compared to the solubility limit in water. This would mean the operator would combine 10000 liters of water and ½ ton of chicken manure in a 15000-liter batch reactor based on the limiting nutrient composition. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the product would be transferred to a filter to remove the solids. The filtrate liquid stream may be analyzed for nutrient deficiencies or unwanted nutrients. The appropriate additives are added to optimize the broth to comply with growth media specifications such as raising the pH with pH buffers, removing heavy metals with chelators, or adding glucose source, to provide energy for the microorganism. The liquid medium may then be processed through a pre-filter and fine filter to remove any undesired solids. Once the broth is optimized, it must be sterilized via autoclave. The broth may be heated to about 121 degrees C. at about 15 psig and held for at least about 15 minutes to ensure sterilization. After the above steps, the product, the cell culture broth or growth media, is ready for packaging and distribution following a final analytical analysis to ensure quality compliance. The solid byproduct left on the filter may be used in anaerobic digestion to produce methane gas.

### PROPHETIC EXAMPLE 3

### Cell Culture Broth/Media from Swine Manure

First, a sample of the source swine manure may be analyzed to determine in what concentration it should be combined with water. Following this analysis, the determination of the proper water to manure ratio can be achieved. Based on the fact that nitrate, phosphates, glucose, and peptides are present at a certain ratio, the manure will be mixed with water based on the limiting nutrient. For example, the amount of phosphate present in the manure may be at an about 20 to 1 ratio compared to the solubility limit in water. This would mean the operator would combine 5000 liters of water and about ¼ ton of chicken manure in a 10000-liter batch reactor based on the limiting nutrient composition. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the product would be transferred to a filter press to remove the solids. The filter press would remove most of the water from the solid byproduct. The filtrate liquid stream may be analyzed for nutrient deficiencies or unwanted nutrients. The appropriate additives are added to optimize the broth to comply with growth media specifications such as raising the pH with pH buffers, removing heavy metals with chelators, or adding glucose source, to provide energy for the microorganism. Once the broth is optimized, it must be sterilized via ultraviolet (UV) sterilization. The broth may be exposed to UV light with a wavelength of 254 nm for a prolonged residence time of over an hour to kill about 99.9999% of the microorganisms. After the above steps, the product, the cell culture broth or growth media, is ready for packaging and distribution following a final analytical analysis to ensure quality compliance. The solid byproduct left on the filter may be used in anaerobic digestion to produce methane gas.

### PROPHETIC EXAMPLE 4

### Cell Culture Broth/Media from Poultry Manure

First, a sample of the source poultry manure may be analyzed to determine in what concentration it should be combined with water. Following this analysis, the determination of the proper water to manure ratio can be achieved. Based on the fact that nitrate, phosphates, glucose, and peptides are present at a certain ratio, the manure will be mixed with water based on the limiting nutrient. For example, the amount of phosphate present in the manure may be at an about 50 to 1 ratio compared to the desired nutrient profile for a dilute broth. This would mean the operator would create a feed that is 50 parts water to 1 part manure. Next, the feed would flow at about 1000 liters per hour with a residence time of about 1 hour through a continuous plug flow reactor (PFR) with static mixer. The reactor may be maintained at room temperature. Optionally, it may be heated up to about 100 degrees C. in order to aid in solubilizing the nutrients in water. Upon leaving the PFR, the product stream would be sent to a continuous decanter to separate the solid and liquid streams. The liquid stream may be analyzed in line for nutrient deficiencies or unwanted nutrients. The appropriate additives are added to optimize the broth to comply with growth media specifications such as raising the pH with pH buffers, removing heavy metals with chelators, or adding glucose source, to provide energy for the microorganism. Once the broth is optimized, it must be sterilized via ultraviolet (UV) sterilization. The broth may be exposed to UV light with a wavelength of 254 nm for a prolonged residence time of over an hour to kill about 99.9999% of the microorganisms. After the above steps, the product, the cell culture broth or growth media, is ready for packaging and distribution following a final analytical analysis to ensure quality compliance. The solid byproduct from the solid stream on the decanter may be used for direct combustion to generate steam or electricity for the operation.

### PROPHETIC EXAMPLE 5

### Cell Culture Broth/Media from Plant Waste

First, a sample of the source plant waste may be analyzed to determine in what concentration it should be combined with water. Following this analysis, the determination of the proper water to manure ratio can be achieved. Based on the fact that nitrate, phosphates, glucose, and peptides are present at a certain ratio, the plant waste will be mixed with water based on the limiting nutrient. For example, the amount of phosphate present in the plant waste may be at an about 50 to 1 ratio compared to the desired nutrient profile for a dilute broth. This would mean the operator would create a feed that is 50 parts water to 1 part manure. Next, the feed would flow at about 1000 liters per hour with a residence time of about 1 hour through a continuous stirred tank reactor (CSTR). The reactor may be maintained at room temperature. Optionally, it may be heated up to about 100 degrees C. in order to aid in solubilizing the nutrients in water. Upon leaving the CSTR, the product stream would be sent to a continuous decanter to separate the solid and liquid streams. The liquid stream may be analyzed in line for nutrient deficiencies or unwanted nutrients. The appropriate additives are added to optimize the broth to comply with growth media specifications such as raising the pH with pH buffers, removing heavy metals with chelators, or adding glucose source, to provide energy for the microorganism. The liquid medium may then be processed through a pre-filter and fine filter to remove any undesired solids. Once the broth is optimized, it must be sterilized via autoclave. The broth may be heated to about 121 degrees C. at about 15 psig and held for at least about 15 minutes to ensure sterilization. After the above steps, the product, the cell culture broth or growth media, is ready for packaging and distribution following a final analytical analysis to ensure quality compliance. The solid byproduct from the solid stream on the decanter may be used for direct combustion to generate steam or electricity for the operation.

### PROPHETIC EXAMPLE 6

### Cell Culture Broth/Media from Poultry Manure

First, the poultry manure is homogenized and broken into smaller particles via a milling/grinding step. Then, a sample of the source poultry manure may be analyzed to determine in what concentration it should be combined with water. Following this analysis, the determination of the proper water to manure ratio can be achieved. Based on the fact that nitrate, phosphates, glucose, and peptides are present at a certain ratio, the manure will be mixed with water based on the limiting nutrient. For example, the amount of phosphate present in the manure may be at an about 20 to 1 ratio compared to the solubility limit in water. This would mean the operator would combine 5000 liters of water and about ¼ ton of chicken manure in a 10000-liter batch reactor based on the limiting nutrient composition. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the product would be transferred to a filter to remove the solids. The broth is then sterilized via autoclave and ultraviolet (UV) sterilization. First, the broth may be heated to about 121 degrees C. at about 15 psig and held for at least about 15 minutes to ensure sterilization. Then, broth may be exposed to UV light with a wavelength of 254 nm for a prolonged residence time of over an hour to kill about 99.9999% of the microorganisms. After the above steps, the product, the cell culture broth or growth media, is ready for packaging and distribution following a final analytical analysis to ensure quality compliance. The solid byproduct left on the filter may be used in anaerobic digestion to produce methane gas.

### PROPHETIC EXAMPLE 7

### Cell Culture Broth/Media from Horse Manure

First, the horse manure is homogenized and broken into smaller particles via a milling/grinding step. Then, a sample of the source horse manure may be analyzed to determine in what concentration it should be combined with water. Following this analysis, the determination of the proper water to manure ratio can be achieved. Based on the fact that nitrate, phosphates, glucose, and peptides are present at a certain ratio, the manure will be mixed with water based on the limiting nutrient. For example, the amount of phosphate present in the manure may be at an about 20 to 1 ratio compared to the solubility limit in water. This would mean the operator would combine 5000 liters of water and about ¼ ton of chicken manure in a 10000-liter batch reactor based on the limiting nutrient composition. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the product would be transferred to a filter to remove the solids. The broth is then sterilized via chemical sterilization. First, the broth may be transferred to a batch reactor where an appropriate chemical can be used to sterilize the broth, such as bleach (sodium hypochlorite). After the above steps, the product, the cell culture broth or growth media, is ready for packaging and distribution following a final analytical analysis to ensure quality compliance. The solid byproduct left on the filter may be used in anaerobic digestion to produce methane gas.

### PROPHETIC EXAMPLE 8

### Cell Culture Broth/Media from Livestock (Cow and Swine) Manure

First, a sample of the source livestock (cow and swine) manure may be analyzed to determine in what concentration it should be combined with water. Following this analysis, the determination of the proper water to swine manure to cow manure ratio can be achieved. Based on the fact that nitrate, phosphates, glucose, and peptides are present at a certain ratios in different manures, the manures would be combined with water at an appropriate ratio to give the desired nutrient profile. For example, cow manure may have a higher percentage of nitrates compared to swine manure, which may have a higher percentage of phosphates and potassium. The operator following this analysis may combine about 5000 liters of water, about 1/4 ton of cow manure and about ⅛ ton of swine manure in a 10000-liter batch reactor based on the limiting nutrient compositions. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the agitation may be turned off and the solids are allowed to gravitationally settle for about 30 to 60 minutes. The product liquid medium may be decanted leaving the solids in the reactor. The liquid medium may then be sterilized in a reactor via chemical sterilization by adding bleach (sodium hypochlorite). The liquid medium may be analyzed for nutrient deficiencies or unwanted nutrients. The appropriate additives are added to optimize the broth to comply with growth media specifications such as raising the pH with pH buffers, removing heavy metals with chelators, or adding glucose source, to provide energy for the microorganism. After the above steps, the product, the cell culture broth or growth media, is ready for packaging and distribution following a final analytical analysis to ensure quality compliance. The solid byproduct left in the reactor may be dried and may be used in a direct combustion process to provide steam or electricity for the process.

### PROPHETIC EXAMPLE 9

### Cell Culture Broth/Media from Poultry and Swine Manure

First, a sample of the source livestock (poultry and swine) manure may be analyzed to determine in what concentration it should be combined with water. Following this analysis, the determination of the proper water to poultry manure to swine manure ratio can be achieved. Based on the fact that nitrate, phosphates, glucose, and peptides are present at a certain ratios in different manures, the manures would be combined with water at an appropriate ratio to give the desired nutrient profile. For example, swine manure may have a higher percentage of nitrates compared to poultry manure, which may have a higher percentage of phosphates and potassium. The operator following this analysis may combine about 5000 liters of water, about ¼ ton of swine manure and about ⅛ ton of poultry manure in a 10000-liter batch reactor based on the limiting nutrient compositions. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the product would be transferred to a filter press to remove the solids. The filter press would remove most of the water from the solid byproduct. Now the broth must be sterilized via ultraviolet (UV) sterilization and microfiltration. The broth may be exposed to UV light with a wavelength of 254 nm for a prolonged residence time of over an hour to kill about 99.9999% of the microorganisms. Following UV sterilization, the broth would undergo microfiltration as an additional sterilization technique. The filtrate liquid stream may be analyzed for nutrient deficiencies or unwanted nutrients. Once the broth is sterilized, it would be optimized. The appropriate additives are added to optimize the broth to comply with growth media specifications such as raising the pH with pH buffers, removing heavy metals with chelators, or adding glucose source, to provide energy for the microorganism. After the above steps, the product, the cell culture broth or growth media, is ready for packaging and distribution following a final analytical analysis to ensure quality compliance. The solid byproduct left on the filter may be used in a direct combustion to provide steam or electricity for the process.

### PROPHETIC EXAMPLE 10

### Cell Culture Broth/Media from Poultry and Swine Manure

First, a sample of the source livestock (poultry and swine) manure may be analyzed to determine in what concentration it should be combined with water. Following this analysis, the determination of the proper water to poultry manure to swine manure ratio can be achieved. Based on the fact that nitrate, phosphates, glucose, and peptides are present at a certain ratios in different manures, the manures would be combined with water at an appropriate ratio to give the desired nutrient profile. For example, swine manure may have a higher percentage of nitrates compared to poultry manure, which may have a higher percentage of phosphates and potassium. The operator following this analysis may combine about 5000 liters of water, about ¼ ton of swine manure and about ⅛ ton of poultry manure in a 10000-liter batch reactor based on the limiting nutrient compositions. Next, the mixture may be agitated for about 30 to 60 minutes at about 150 to 200 rpm under turbulent conditions. The reactor may be maintained at room temperature. Optionally, it may be heated up to about 100 degrees C. in order to aid in solubilizing the nutrients in water. Next, the product would be transferred to a filter to remove the solids. The filtrate liquid stream may be analyzed for nutrient deficiencies or unwanted nutrients. The liquid medium may then be processed through a pre-filter and fine filter to remove any undesired solids. Once the broth is optimized, it must be sterilized via autoclave. The broth may be heated to about 121 degrees C. at about 15 psig and held for at least about 15 minutes to ensure sterilization. The filtrate liquid stream may be analyzed for nutrient deficiencies or unwanted nutrients. Once the broth is sterilized, it would be optimized. The appropriate additives are added to optimize the broth to comply with growth media specifications such as raising the pH with pH buffers, removing heavy metals with chelators, or adding glucose source, to provide energy for the microorganism. After the above steps, the product, the cell culture broth or growth media, is ready for packaging and distribution following a final analytical analysis to ensure quality compliance. The solid byproduct left on the filter may be used in anaerobic digestion to produce methane gas.

While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1. A method for converting organic waste material into a cell culture broth or growth media comprising:
mixing an organic waste material with one or more solvent under substantially turbulent conditions to create a mixture of liquids and solids;
separating the mixture of liquids and solids into a liquid stream and solid stream; and
sterilizing the liquid stream,
wherein the cell culture broth or growth media comprises the sterilized liquid stream.

2. The method of claim 1, wherein the culture broth and/or growth media is suitable for the propagation, culture, fermentation, or maintenance of bacteria, algae, yeast, synthetically derived microorganisms, insect cells, plant cells, or mammalian cells.

3. The method of claim 1, wherein the organic waste material is selected from the group consisting of livestock manure, bedding, litter, human organic waste, plant waste and mixtures thereof.

4. The method of claim 3, wherein the organic waste material is processed.

5. The method of claim 4, wherein the organic waste material is processed by milling, composting, pyrolysis, autoclaving, gasification, anaerobic digestion, combustion, and combinations thereof.

6. The method of claim 1, wherein the organic waste material comprises plant waste free of livestock manure, bedding, litter, and human organic waste.

7.-10. (canceled)

11. The method of claim 1 further comprising:
analyzing the organic waste material for chemical composition prior to mixing;
analyzing the liquid stream and the solid stream for chemical composition after separating the mixture of liquids and solids into a liquid stream and solid stream;
optimizing the composition of the liquid stream depending on the desired use of the cell culture broth or growth media; and
optionally selecting one or more solvent based on the chemical composition of the organic waste material prior to mixing the organic waste material with the one or more solvent.

12. The method of claim 11, wherein the step of optimizing comprises one or more of (a) adding nutrients or chemicals to the liquid stream, (b) removing and/or deactivating excess nutrients or chemicals from the liquid stream, (c) removing and/or deactivating antibiotics and other growth harming substances from the liquid stream and combinations thereof.

13.-16. (canceled)

17. The method of claim 1, wherein the one or more solvent is water, alcohol or an organic solvent.

18. The method of claim 1, wherein the step of mixing comprises one or more of the following:
a residence time from about 15 minutes to about 3 hours;
heating organic waste material in one or more solvent;
heating up to about 100 degrees C.;
mixing under turbulent conditions;
mixing under conditions having a Reynolds number (Re) of greater than 2000;
mixing under transitional conditions;
agitation; or
agitation for about 30 to 60 minutes at about 150 to 200 rpm.

19.-24. (canceled)

25. The method of claim 1, wherein the step of separating the mixture of liquids and solids into a liquid stream and solid stream comprises filtration, gravitational settling, decanting, centrifugation, or combinations thereof.

26. The method of claim 1, wherein the liquid stream comprises nutrients, and one or more solvent.

27. The method of claim 1, wherein the step of sterilizing the liquid stream comprises filtration, irradiation, chemical treatment, heating, pressurization, or combinations thereof.

28. The method of claim 27, wherein the step of sterilizing the liquid stream comprises heating the liquid stream to a temperature of about 121 degrees C. at 15 psig and maintaining the temperature for at least 15 minutes.

29. The method of claim 1 further comprising anaerobic digestion of the solid stream, gasification of the solid stream, combustion of the solid stream, pyrolysis of the solid stream or combinations thereof.

30. The method of claim 1 further comprising reusing the solid stream in the process by mixing the solid stream with the organic materials and optionally anaerobic digestion of the solid stream prior to mixing with the organic materials.

31. The method of claim 1, wherein the solid stream is suitable for use as a plant fertilizer.

32. (canceled)

33. The method of claim 1 further comprising one or more of the following steps:
adding nutrients or chemicals to the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells without the nutrients or chemicals;
removing and/or deactivating excess nutrients or chemicals from the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells with the nutrients or chemicals;
removing and/or deactivating antibiotics and other growth harming substances from the liquid stream to increase the growth rate of bacteria, algae, yeast, synthetically derived microorganisms, insect cells, plant cells, or mammalian cells as compared to a growth rate of the bacteria, algae, yeast, synthetically derived microorganisms, insect cells, plant cells, or mammalian cells with the antibiotics and other growth harming substances; or
the combinations thereof.

34.-35. (canceled)

36. The method of claim 1, wherein the cell culture broth or growth media is supplemented to achieve optimal growth conditions of bacteria, algae, yeast, synthetically derived microorganisms, insect cells, plant cells, or mammalian cells.

37. A cell culture broth or growth media produced by the method of claim 1.

38. (canceled)
